(19) United States
(12) Patent Application Publication
(10) Pub. No.: US 2005/0234115 A1
(43) Pub. Date: Oct. 20, 2005

(54) Dosage forms and methods of treatment using VEGFR inhibitors

(75) Inventors: Diane I. Healey, Madison, CT (US); Dennis A. Noe, Madison, CT (US); James J. O'Leary, Mystic, CT (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 11/109,904

(22) Filed: Apr. 20, 2005

Related U.S. Application Data
(60) Provisional application No. 60/564,286, filed on Apr. 20, 2004.

(51) Int. Cl. A61K 31/425

(52) U.S. Cl. 514/372

(57) ABSTRACT

The invention provides dosage forms of a compound of formula 1: 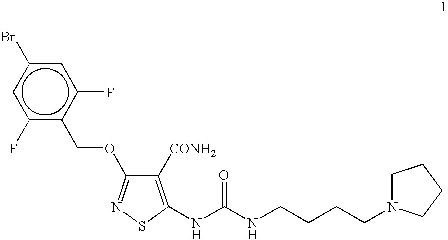 or pharmaceutically acceptable salts, solvates or prodrugs thereof. The invention further provides methods of treating hyperproliferative diseases, such as cancers, by administering the dosage forms to a mammal.

### BACKGROUND OF THE INVENTION

This invention relates to VEGFR inhibitors that are useful in the treatment of hyperproliferative diseases, such as cancer, in mammals. More particularly, this invention relates to dosage forms of 3-(4-Bromo-2,6-difluoro-benzyloxy)-5-[3-(4-pyrrolidin-1-yl-butyl)-ureido]-isothiazole-4-carboxylic acid amide, represented by formula 1 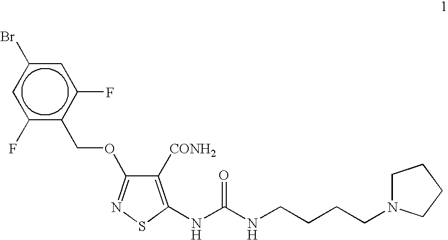
its pharmaceutically acceptable salt, solvate or prodrug thereof, or a mixture thereof, useful in the treatment of hyperproliferative diseases, especially humans, and to pharmaceutical compositions containing such compounds.

The compound of formula 1 is an anti-angiogenic small molecule inhibitor of the tyrosine kinase activity of the vascular endothelial growth factor receptor 2 (VEGFR-2). As such, it inhibits VEGF stimulated VEGFR-2 autophosphorylation in whole cells, and inhibits endothelial cell proliferation. Angiogenesis is necessary for the growth and metastasis of all solid tumors, in that newly formed blood vessels provide nutrients for growing tumors. VEGF is highly overexpressed in a number of tumors. The therapeutic objective of the compound of formula 1 is to inhibit angiogenesis and thereby prevent tumor growth by inhibition of VEGFR-2 tyrosine kinase (TK) activity. The compound is approximately 250× to 1000× selective for VEGFR-2 and bFGF relative to the concentrations required to inhibit the platelet derived growth factor-β (PDGFR-β), the epidermal growth factor receptor (EGFR) and the insulin receptor (IR) tyrosine kinases.

VEGF inhibitors are described in, for example in WO 99/62890 (published Dec. 9, 1999), U.S. Pat. No. 6,235,764 (issued May 22, 2001) and U.S. Pat. No. 6,548,526 (issued Apr. 15, 2003); WO 01/95353 (published Dec. 13, 2001), WO 02/44158 (published Jun. 6, 2002), WO 04/017964 (published Mar. 4, 2004), WO 99/24440 (published May 20, 1999), WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998). Other examples of some specific VEGF inhibitors include IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.).

### SUMMARY OF THE INVENTION

The invention provides dosage forms and methods of treatment using a compound of formula 1: 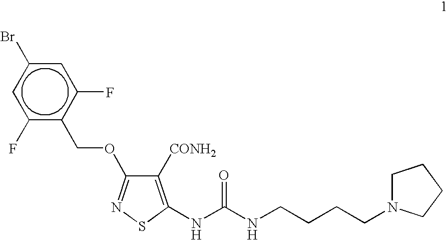
which can be systematically named as 3-(4-Bromo-2,6-difluoro-benzyloxy)-5-[3-(4-pyrrolidin-1-yl-butyl)-ureido]-isothiazole-4-carboxylic acid amide, a pharmaceutically acceptable salt, solvate or prodrug thereof, or a mixture thereof.

In an especially preferred embodiment, the pharmaceutically acceptable salt is a hydrochloride salt of the compound of formula 1. The hydrochloride salt can be represented by the compound of formula 2 set forth below:

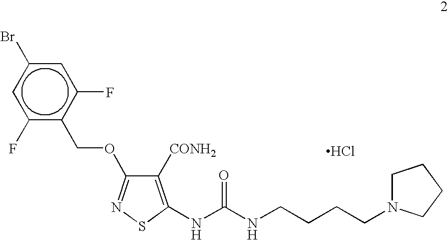

In one embodiment, the invention provides a dosage form for administration to a mammal, the dosage form comprising the compound of formula 1, a pharmaceutically acceptable salt, solvate or prodrug thereof, or a mixture thereof, in an amount effective to provide a 24-hour AUC value, (i.e., dosing interval AUC value) of no more than about 30000 ng·hr/mL of the compound of formula 1 or active metabolites thereof, after multiple daily (i.e., QD) administration to the mammal.

In specific aspects of this embodiment, the 24-hour AUC value is from about 1000 to about 30000 ng·hr 1 mL, and in one embodiment from about 1200 to about 28000 ng·hr/mL, and in one embodiment from about 1440 to about 26000 ng·hr/mL, and in one embodiment from about 2000 to about 25000 ng·hr/mL.

In another embodiment, the dosage form is an oral dosage form. In another embodiment, the dosage form is a tablet or a capsule.

In another embodiment, the invention provides a dosage form comprising the compound of formula 1, a pharmaceutically acceptable salt, solvate or prodrug thereof, or a mixture thereof, in an amount of no more than about 300 mg.

In specific aspects of this embodiment, the dosage form is from about 125 to about 300 mg., in one embodiment from about 150 to about 280 mg., in one embodiment from about 180 to about 260 mg., and in one embodiment about 250 mg.

In another aspect of this embodiment, the dosage form is an oral dosage form, and in another embodiment, the dosage form is a tablet or a capsule.

In another embodiment, the invention provides a method of treating a hyperproliferative disorder in a mammal which comprises administering to said mammal in need of such treatment the compound of formula 1, a pharmaceutically acceptable salt, solvate or prodrug thereof, or a mixture thereof, in an amount effective to provide a 24-hour AUC value, (i.e., dosing interval AUC value) of no more than about 30000 ng·hr/mL of the compound of formula 1 or active metabolites thereof, after multiple daily (i.e., QD) administration to the mammal.

In specific aspects of this embodiment, the 24-hour AUC values are the same as those set forth above. In another aspect of this embodiment, the compound is administered orally and in another aspect of this embodiment, it is administered at a dosage frequency of at least once per day.

In another aspect of this embodiment, the hyperproliferative disorder is cancer, including but not limited to brain, squamous cell, bladder, gastric, pancreatic, breast (including metastatic breast cancer), head, neck, oesophageal, prostate, colorectal, lung (including non-small cell lung), renal, kidney, ovarian, gynecological and thyroid cancer. In another aspect of this embodiment, the cancer is a solid tumor. In another aspect of this embodiment, the hyperproliferative disorder is non-cancerous, such as benign hyperplasia of the skin or prostrate.

In another aspect of this embodiment, the method of treating the hyperproliferative disorder in the mammal further comprises administering to said mammal in need of such treatment, either simultaneously or sequentially with the compound of claim 1, a pharmaceutically acceptable salt, solvate or prodrug thereof, or a mixture thereof, a therapeutically effective amount of at least one compound selected from the group consisting of taxane derivatives (such as paclitaxel and docetaxel) and platinum coordination complexes selected from the group consisting of cisplatin, carboplatin, tetraplatin, and topotecan. In one preferred aspect of this embodiment, the taxane is paclitaxel and the platinum coordination complex is carboplatin.

In another embodiment, the invention provides a method of treating a hyperproliferative disorder in a mammal which comprises administering to said mammal in need of such treatment the compound of formula 1, a pharmaceutically acceptable salt, solvate or prodrug thereof, or a mixture thereof, a pharmaceutically acceptable salt, solvate or prodrug thereof, or a mixture thereof, in an amount of no more than about 300 mg per dose.

In specific aspects of this embodiment, this method utilizes those dosage amounts in milligrams as set forth above. In another aspect of this embodiment, the compound is administered orally and in another aspect of this embodiment, it is administered at a dosage frequency of at least once per day. In another aspect of this embodiment, the hyperproliferative disorder is cancer, including the various types set forth above, and in another aspect, the hyperproliferative disorder is non-cancerous, such as benign hyperplasia of the skin or prostrate.

In another aspect of this embodiment, the method of treating the hyperproliferative disorder in the mammal further comprises administering to said mammal in need of such treatment, either simultaneously or sequentially with the compound of claim 1, a pharmaceutically acceptable salt, solvate or prodrug thereof, or a mixture thereof, a therapeutically effective amount of at least one compound selected from the group consisting of taxane derivatives (such as paclitaxel and docetaxel) and platinum coordination complexes selected from the group consisting of cisplatin, carboplatin, tetraplatin, and topotecan. In one preferred aspect of this embodiment, the taxane is paclitaxel and the platinum coordination complex is carboplatin.

In another embodiment, the invention provides a method of treating a hyperproliferative disorder in a mammal which comprises administering to said mammal in need of such treatment a dose of about 125 mg/day once a day (QD) to about 300 mg/day once a day of the compound of formula 1, a pharmaceutically acceptable salt, solvate or prodrug thereof, or a mixture thereof.

In specific aspects of this embodiment, the dose is from about 150 mg/day QD to about 280 mg/day QD, and in another aspect from about 180 mg/day QD to about 260 mg/day QD, and in another aspect about 250 mg/day QD. In another aspect of this embodiment, the compound is administered orally, and in another aspect the hyperproliferative disorder is cancer, including the various types set forth above.

In another aspect of this embodiment, the method of treating the hyperproliferative disorder in the mammal further comprises administering to said mammal in need of such treatment, either simultaneously or sequentially with the compound of claim 1, a pharmaceutically acceptable salt, solvate or prodrug thereof, or a mixture thereof, a therapeutically effective amount of at least one compound selected from the group consisting of taxane derivatives (such as paclitaxel and docetaxel) and platinum coordination complexes selected from the group consisting of cisplatin, carboplatin, tetraplatin, and topotecan. In one preferred aspect of this embodiment, the taxane is paclitaxel and the platinum coordination complex is carboplatin.

In another embodiment, the invention provides a method of treating a hyperproliferative disorder in a mammal which comprises administering to said mammal in need of such treatment the compound of formula 1, a pharmaceutically acceptable salt, solvate or prodrug thereof, or a mixture thereof, in an amount of no more than about 300 mg per dose and paclitaxel in an amount of no more than about 250 mg/m², said paclitaxel being administered once every three weeks per cycle.

In specific aspects of this embodiment, the amount of the compound of formula 1, a pharmaceutically acceptable salt, solvate or prodrug thereof, or a mixture thereof, is from about 125 to about 300 mg, wherein the amount of paclitaxel is from about 200 to about 250 mg/m²; in another aspect, the amount of the compound of formula 1, a pharmaceutically acceptable salt, solvate or prodrug thereof, or a mixture thereof, is from about 150 to about 280 mg, wherein the amount of paclitaxel is from about 210 to about 240 mg/m²; in another aspect, the amount of the compound of formula 1, a pharmaceutically acceptable salt, solvate or prodrug thereof, or a mixture thereof, is from about 180 to about 260 mg, wherein the amount of paclitaxel is from about 220 to about 230 mg/m²; and in another aspect, the amount of the compound of formula 1, a pharmaceutically acceptable salt, solvate or prodrug thereof, or a mixture thereof, is about 200 mg, wherein the amount of paclitaxel is about 225 mg/m².

In another aspect of this embodiment, the compound of formula 1 is administered orally and the paclitaxel is administered intravenously. In another aspect of this embodiment, the compound of formula 1, a pharmaceutically acceptable salt, solvate or prodrug thereof, or a mixture thereof, is administered once a day (QD). In another aspect of this embodiment, the hyperproliferative disorder is cancer, including the various types set forth above.

In another embodiment, the invention provides a method of treating a hyperproliferative disorder in a mammal which comprises administering to said mammal in need of such treatment the compound of formula 1, a pharmaceutically acceptable salt, solvate or prodrug thereof, or a mixture thereof, in an amount of no more than about 300 mg per dose and paclitaxel in an amount of no more than about 80 mg/m², said paclitaxel being administered once every week per cycle.

In specific aspects of this embodiment, the amount of the compound of formula 1, a pharmaceutically acceptable salt, solvate or prodrug thereof, or a mixture thereof, is from about 125 to about 300 mg, wherein the amount of paclitaxel is from about 30 to about 75 mg/m²; in another aspect, the amount of the compound of formula 1, a pharmaceutically acceptable salt, solvate or prodrug thereof, or a mixture thereof, is from about 150 to about 280 mg, wherein the amount of paclitaxel is from about 40 to about 70 mg/m²; in another aspect, the amount of the compound of formula 1, a pharmaceutically acceptable salt, solvate or prodrug thereof, or a mixture thereof, is from about 180 to about 260 mg, wherein the amount of paclitaxel is from 50 to 60 mg/m²; and in another aspect, the amount of the compound of formula 1, a pharmaceutically acceptable salt, solvate or prodrug thereof, or a mixture thereof, is about 200 mg, wherein the amount of paclitaxel is from about 50 mg/m² to about 60 mg/m².

In another aspect of this embodiment, the compound of formula 1, a pharmaceutically acceptable salt, solvate or prodrug thereof, or a mixture thereof, is administered orally and the paclitaxel is administered intravenously. In another aspect of this embodiment, the compound of formula 1, a pharmaceutically acceptable salt, solvate or prodrug thereof, or a mixture thereof, is administered once a day (QD). In another aspect of this embodiment, the hyperproliferative disorder is cancer, including the various types set forth above.

In another embodiment, the invention provides a method of treating a hyperproliferative disorder in a mammal which comprises administering to said mammal in need of such treatment the compound of formula 1, a pharmaceutically acceptable salt, solvate or prodrug thereof, or a mixture thereof, in an amount of no more than about 300 mg per dose and carboplatin in an amount effective to provide an AUC value of no more than about 8 ng·hr/mL of carboplatin after administration to the mammal, said carboplatin being administered once every three weeks per cycle.

In specific aspects of this embodiment, the amount of the compound of formula 1, a pharmaceutically acceptable salt, solvate or prodrug thereof, or a mixture thereof, is from about 125 to 300 mg, wherein the amount of carboplatin is an amount effective to provide an AUC value of from about 4 to about 8 ng·hr/mL; in another aspect, the amount of the compound of formula 1, a pharmaceutically acceptable salt, solvate or prodrug thereof, or a mixture thereof, is from about 150 to about 280 mg, wherein the amount of carboplatin is an amount effective to provide an AUC value of from about 5 to 7 ng·hr/mL; in another aspect, the amount of the compound of formula 1, a pharmaceutically acceptable salt, solvate or prodrug thereof, or a mixture thereof, is from about 180 to about 260 mg, wherein the amount of carboplatin is an amount effective to provide an AUC value of from about 5 to about 7 ng·hr/mL; and in another aspect, the amount of the compound of formula 1, a pharmaceutically acceptable salt, solvate or prodrug thereof, or a mixture thereof, is about 200 mg, wherein the amount of carboplatin is an amount effective to provide an AUC value of about 6 ng·hr/mL.

In another aspect of this embodiment, the compound of formula 1, a pharmaceutically acceptable salt, solvate or prodrug thereof, or a mixture thereof, is administered orally and the carboplatin is administered intravenously. In another aspect of this embodiment, the compound of formula 1, a pharmaceutically acceptable salt, solvate or prodrug thereof, or a mixture thereof, is administered once a day (QD). In another aspect of this embodiment, the hyperproliferative disorder is cancer, including the various types set forth above.

In another embodiment, the invention provides a method of treating a hyperproliferative disorder in a mammal which comprises administering to said mammal in need of such treatment the compound of formula 1, a pharmaceutically acceptable salt, solvate or prodrug thereof, or a mixture thereof, in an amount of no more than about 300 mg per dose, paclitaxel in an amount of no more than about 250 mg/m², and carboplatin in an amount effective to provide an AUC value of no more than about 8 ng·hr/mL of carboplatin after administration to the mammal, each of said paclitaxel and carboplatin being administered once every three weeks per cycle.

In specific aspects of this embodiment, the amount of the compound of formula 1, a pharmaceutically acceptable salt, solvate or prodrug thereof, or a mixture thereof, is from about 125 to about 300 mg, wherein the amount of paclitaxel is from about 200 to about 250 mg/m², and wherein the amount of carboplatin is in an amount effective to provide an AUC value of from about 4 to about 8 ng·hr/mL; in another aspect, the amount of the compound of formula 1, a pharmaceutically acceptable salt, solvate or prodrug thereof, or a mixture thereof, is from about 150 to about 280 mg, wherein the amount of paclitaxel is from about 210 to about 240 mg/m², and wherein the amount of carboplatin is in an amount effective to provide an AUC value of from about 5 to about 8 ng·hr/mL; in another aspect, the amount of the compound of formula 1, a pharmaceutically acceptable salt, solvate or prodrug thereof, or a mixture thereof, is from about 180 to about 260 mg, wherein the amount of paclitaxel is from about 210 to about 240 mg/m², and wherein the amount of carboplatin is in an amount effective to provide an AUC value of from about 5 to about 8 ng·hr/mL; and in another aspect, the amount of the compound of formula 1, a pharmaceutically acceptable salt, solvate or prodrug thereof, or a mixture thereof, is about 200 mg, wherein the amount of paclitaxel is about 225 mg/m², and wherein the amount of carboplatin is in an amount effective to provide an AUC value of about ng·hr/mL.

In another aspect of this embodiment, the compound of formula 1, a pharmaceutically acceptable salt, solvate or prodrug thereof, or a mixture thereof, is administered once a day (QD). In another aspect of this embodiment, the hyperproliferative disorder is cancer, including the various types set forth above.

As used herein, unless indicated otherwise, the term “AUC” means “area under the plasma concentration versus time curve”. The 24-hour AUC value refers to dosing interval AUC value for once a day (QD) dosing.

“Hyperproliferative disorder”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Salt forms of 3-(4-Bromo-2,6-difluoro-benzyloxy)-5-[3-(4-pyrrolidin-1-yl-butyl)-ureido]-isothiazole-4-carboxylic acid amide and their method of production is disclosed in International Publication WO 02/44158 (published Jun. 6, 2002).

The term “prodrug”, as used herein, unless otherwise indicated, means compounds that are drug precursors, which following administration, release the drug in vivo via some chemical or physiological process (e.g., a prodrug on being brought to the physiological pH is converted to the desired drug form). Prodrugs include compounds wherein an amino acid residue, or a polypeptide chain of two or more (e.g., two, three or four) amino acid residues is covalently joined through an amide or ester bond to a free amino, hydroxy or carboxylic acid group of the compound of formula 1. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs are also encompassed. For instance, free carboxyl groups can be derivatized as amides or alkyl esters. The amide and ester moieties may incorporate groups including but not limited to ether, amine and carboxylic acid functionalities. Free hydroxy groups may be derivatized using groups including but not limited to hemisuccinates, phosphate esters, dimethylaminoacetates, and phosphoryloxymethyloxycarbonyls, as outlined in D. Fleisher, R. Bong, B. H. Stewart, Advanced Drug Delivery Reviews (1996) 19, 115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in R. P. Robinson et al., J. Medicinal Chemistry (1996) 39, 10.

The subject invention also includes isotopically-labelled compounds, which are identical to those recited in Formula 1, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of Formula 1 of this invention and prodrugs thereof can generally be prepared by carrying out the procedures described for the non-labeled compound, substituting a readily available isotopically labeled reagent for a non-isotopically labeled reagent.

Each of the patents, patent applications, published International applications, and scientific publications referred to in this patent application is incorporated herein by reference in its entirety.

### DETAILED DESCRIPTION OF THE INVENTION

The compound of formula 1 can be prepared as described in WO 99/62890, and U.S. Pat. Nos. 6,235,724 and 6,548,526. Certain starting materials may be prepared according to methods familiar to those skilled in the art and certain synthetic modifications may be done according to methods familiar to those skilled in the art.

The compound of formula 1 is capable of forming a wide variety of different salts with various inorganic and organic acids. Although such salts must be pharmaceutically acceptable for administration to mammals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Administration of the compound of formula 1 can be effected by any method that enables delivery of the compound to the site of action. These methods include oral routes, intraduodenal routes, parenteral injection (including intravenous, subcutaneous, intramuscular, intravascular or infusion), topical, and rectal administration.

The compound may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository. The compound may be in unit dosage forms suitable for single administration of precise dosages. Preferably, dosage forms include a conventional pharmaceutical carrier or excipient and the compound of formula 1 as an active ingredient. In addition, dosage forms may include other medicinal or pharmaceutical agents, carriers, adjuvants, etc.

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

In preferred embodiments of the dosage forms of the invention, the dosage form is an oral dosage form, more preferably, a tablet or a capsule.

In preferred embodiments of the methods of the invention, the compound of formula 1 is administered orally, such as, for example, using an oral dosage form as described herein.

The methods include administering the compound of formula 1 using any desire dosage regimen. In one specific embodiment, the compound is administered once per day (quaque die, or QD).

Methods of preparing various dosage forms with a specific amount of the compound of formula 1 are known, or will be apparent, to those skilled in this art. For examples, see Remington's Pharmaceutical Sciences, Mack Publishing Company, Easter, Pa., 15th Edition (1975).

AUC values can be determined by directly measuring blood plasma concentrations of the compound of formula one or active metabolites thereof, such as by liquid chromatography-tandem mass spectrometry (LC-MS/MS), at various time intervals, and calculating the area under the plasma concentration versus time curve. With daily (i.e., QD) dosing, the 24-hour AUC value (i.e., the dosing interval AUC value) is a standard measure of systemic exposure to the compound. Sodium bisulfite is added as a stabilizer in the reconstitution solution for preparation of concentration standards.

The examples and preparations provided below further illustrate and exemplify the dosage forms and methods of the present invention. It is to be understood that the scope of the present invention is not limited in any way by the scope of the following examples.

### EXAMPLE 1

### Preparation of Free base of 3-(4-bromo-2,6-difluoro-benzyloxy)-5-[3-(4-pyrrolidin-1-yl-butyl)-ureido]-isothiazole-4-carboxylic acid amide

The free base of 3-(4-bromo-2,6-difluoro-benzyloxy)-5-[3-(4-pyrrolidin-1-yl-butyl)-ureido]-isothiazole-4-carboxylic acid amide is prepared according to the procedure described in WO 99/62890 (published Dec. 9, 1999). The compound was prepared from [3-(4-bromo-2,6-difluoro-benzyloxy)-4-carbamoyl-isothiazol-5-yl]-carbamic acid phenyl ester and 4-pyrrolidin-1-yl-butylamine by a procedure analogous to the procedure set forth below to prepare 3-(2,6-Difluoro-4-methyl-benzyloxy)-5-{3-[3-(4-methyl-piperazin-1-yl)-propyl]-ureido}isothiazole-4-carboxylic acid amide. The free base of 3-(4-bromo-2,6-difluoro-benzyloxy)-5-[3-(4-pyrrolidin-1-yl-butyl)-ureido]-isothiazole-4-carboxylic acid had the following characteristics: MS (APCl, m/z): 532 and 534 [M+H]⁺. Mp 208° C. (DSC). Characteristic X-ray powder diffraction peaks (2-theta, [% relative intensity]): 9.314 [100.0], 11.356 [44.8], 15.897 [49.6], 22.059 [84.5], 22.520 [63.3], 22.726 [70.0], 23.927 [67.6], 24.307 [60.5], 25.310 [64.8], and 26.551 [86.6].

### 3-(2,6-Difluoro-4-methyl-benzyloxy)-5-{3-[3-(4-methyl-piperazin-1-yl)-propyl]-ureido}-isothiazole-4-carboxylic acid amide

To a suspension of [4-Carbamoyl-3-(2,6-difluoro-4-methyl-benzyloxy)-isothiazol-5-yl]-carbamic acid phenyl ester (125 mg, 0.298 mmol) in THF (1 mL) was added 1-(3-aminopropyl)-4-methylpiperazine (70 mg, 0.45 mmol). The mixture was shaken at 50° C. overnight, cooled to ambient temperature, and loaded directly onto a radial chromatograph followed by elution with chloroform/methanol/concentrated ammonium hydroxide (50/5/1) to afford a white solid (121 mg, 0.251 mmol, 84%). ¹H NMR (400 MHz, CDCl₃) δ 1.72 (t, J=5.81 Hz, 2H), 2.20-2.85 (m, 10H), 2.28 (s, 3H superimposed on multiplet from 2.20-2.85), 2.35 (s, 3H superimposed on multiplet from 2.20-2.85), 3.39 (t, J=5.4 Hz, 2H), 5.51 (s, 2H), 5.74 (broad s, 1H), 6.74 (d, J=8.3 Hz, 2H), 7.05 (s, 1H), 7.58 (broad s, 1H), 11.01 (broad s, 1H) ppm; MS (APCl, m/z): 483 [M+H]⁺.

### EXAMPLE 2

### Hydrochloride salt of 3-(4-bromo-2,6-difluoro-benzyloxy)-5-[3-(4-pyrrolidin-1-yl-butyl)-ureido]-isothiazole-4-carboxylic acid amide

The compound 3-(4-bromo-2,6-difluoro-benzyloxy)-5-[3-(4-pyrrolidin-1-yl-butyl)-ureido]-isothiazole-4-carboxylic acid amide (500 mg, 0.939 mmol) was dissolved in EtOH (20 mL) at reflux, allowed to cool to ambient temperature and treated with HCl (0.94 mL of a 1.0 M solution in Et₂O) while swirling flask. The mixture was then shaken gently with heating at 50° C. for 3 hours and at ambient temperature for 3 days. The solid was filtered, dried under high vacuum to afford a white solid (468 mg, 0.823 mmol, 82%). Melting point 230° C. (DSC). Hygroscopicity: 1% (by weight)) at 90% relative humidity at ambient temperature (RH). Characteristic X-ray powder diffraction peaks (2-theta, [% relative intensity]): 8.623 [90.7], 12.121 [38.9], 17.298 [95.2], 23.397 [44.7], 23.944 [51.7], 24.119 [62.7], 24.873 [55.7], 25.948 [100], and 28.821 [39.6].

### EXAMPLE 3

A phase 1/2 study examined the safety and PK of escalating oral QD doses of the hydrochloride salt of the compound of formula 1, viz., the hydrochloride salt of 3-(4-Bromo-2,6-difluoro-benzyloxy)-5-[3-(4-pyrrolidin-1-yl-butyl)-ureido]-isothiazole-4-carboxylic acid amide (i.e., compound of formula 2 shown above), given in combination with paclitaxel (P) (225 mg m²) and carboplatin (C) (AUC=6) administered in 21 day cycles to chemotherapy naïve patients with PS 0-1 stage IIIb or IV NSCLC with or without stable brain mets and PS 0-1. Anti-tumor activity was also assessed. 29 patients received a median of 2+ cycles (range 1-13) of study therapy. In this setting, the maximum tolerable dose for the compound of formula 2 was 200 mg/d.

| | Compound of | | |
|---|---|---|---|
| Cohort | formula 2 | #Patients | |
| # | Dose (mg/d) | Treated | Dose Limiting Toxicity (#Pts) |
| 1 | 100 | 8 | Grade 3 Supraventricular |
| | | | tachycardia (1) |
| 2 | 150 | 8 | Gr. 3 Stomatitis (1) |
| 3 | 200 | 8 | Gr. 3 Rash (1) |
| 4 | 250 | 5 | Gr. 3 Diarrhea despite inter- |
| | | | vention (1) Gr. 3 Rash (1) |

Preliminary safety data reveal the most common treatment emergent adverse events to be diarrhea, fatigue, nausea, neuropathy, emesis, alopecia, dyspnea and arthralgia/myalgia. The hydrochloride salt of the compound of formula 1 (i.e., compound of formula 2) has a half-life of approximately 32 hours and, at doses ≧150 mg QD, achieves plasma concentrations within the range associated with anti-angiogenesis in preclinical models. In the 24 patients evaluable for response, a 20% objective response rate and a 33% progressive disease rate were observed. An additional 6 patients were enrolled to the expansion portion of the trial. In this group, confirmation of the safety of the compound of formula 2 at 200 mg when given with P/C is ongoing. The conclusion is that 200 mg, QD oral of the compound of formula 2 can be safely combined with standard doses of P/C. This trial supports investigation in the Phase II setting.

### EXAMPLE 4

In a phase 1 study, fifty five (55) patients were treated with the compound of formula 2 as a single agent over a dose range from 35 mg PO (oral dose) for 14 days to 400 mg PO per day continuously. Overall, 25 male and 30 female patients were treated. Patient malignancies include: NSCL (11 patients), renal and urothelial (7 patients), breast (6 patients), colorectal (7 patients), esophageal (3 patients), pancreatic (2 patients), prostate (3 patients), melanoma (2 patients), head and neck (3 patients), soft tissue sarcoma (3 patients), other (8 patients). Forty-two patients had prior chemotherapy. Three patients starting at 300 mg/d had dose reductions to 225 mg/d; 2 reductions were for grade 3 hypertension (HTN), graded by the Common Toxicity Criteria (CTC version 2), occurring between days 15 and 18; 1 additional reduction was as per the patient's request after an episode of hemoptysis in this patient with renal cell carcinoma and lung metastasis. CTC Grade 3 hypertension was initially considered dose limiting at doses of the compound of formula 2 of 300 mg/d, occurring in 2 of 6 patients. Both patients returned to CTC grade 1 or better within 7 days of holding study drug. Because hypertension has been reported in studies with other VEGFR inhibitors, and mounting evidence suggests that hypertension might be an expected toxicity seen with this class of compound, a hypertension management plan was instituted in the Phase I protocol to allow revisiting the 300 mg dose level.

Other adverse events related to the compound of formula 2 reported to date in the Phase 1 protocol have included the following: CTC grade 1 to 2 diarrhea at all doses >110 mg/d, 3 reports of grade 3 diarrhea, 1 at 225 and 2 at 300 mg. Two of the 3 reports of grade 3 diarrhea were not considered to be dose limiting due to their short duration and lack of prophylaxis. One patient receiving 300 mg/d had a single episode of incontinence after approximately 2 weeks of therapy. Another patient receiving 225 mg/d experienced CTC grade 3 diarrhea of short duration in cycle 2. That patient went on to complete 4 additional cycles of therapy with 225 mg/d of the compound of formula 2 with grade 0-1 diarrhea. The diarrhea does appear to be dose related but does not increase in severity with continued therapy. One patient was reported to have grade 3 colitis at 300 mg. This patient underwent a colonoscopy, which revealed characteristics compatible with ischemic colitis. This patient discontinued study drug and the colitis resolved within 2 weeks. Other adverse events of the gastrointestinal tract occurring in 2 or more patients included nausea, taste disturbance, anorexia and mouth dryness (all grade 1). Given the few number of patients reporting these events, it is difficult to ascribe causality to the compound of formula 2. Dermatologic events reported by more than one patient across all dose levels included grade 2 venostasis rash, which occurred in cycle 2, and grade 1 pallor and shingles. Only 1 of 10 patients treated at the 225 mg/d dose level experienced dose limiting toxicity. This dose limiting event was bleeding of a gastrointestinal tumor, within approximately 2 weeks of starting therapy, which necessitated the removal of the patient from the study. Although bleeding is not atypical for this type of tumor, a causal association with the compound of formula 2 could not be excluded. None of the 4 patients treated at the 250 mg/d dose level experienced dose limiting toxicities.

Of the 29 patients for whom tumor response data is available, 9 patients have stable disease beyond 2 cycles (more than 8 weeks) and 2 patients had stable disease beyond 4 cycles (more than 16 weeks).

There were no significant toxicities reported in the 3 additional patients treated at the 300 mg dose level. One patient at the 350 mg dose level with metastatic NSCLC, 4 days following the removal of the patient from the study for disease progression, was hospitalized for treatment for pulmonary embolism. The patient subsequently died 11 days following the last dose of study drug. While it was generally agreed that the patient was at high risk for developing pulmonary embolism due to his underlying disease, the investigator could not exclude a possible causal relationship between the pulmonary emboli and study drug. Two other patients completed 28 days of treatment at 350 mg without report of significant toxicity. The first two patients treated at the 400 mg dose level experienced dose limiting headache and for this reason, 400 mg was declared the maximum administered dose. One of these patients developed rapidly progressive disease and was removed from the study before she could resume treatment at a reduced dose. The second patient was dose reduced to 350 mg initially, but developed grade 3 fatigue, which required further dose reduction to 300 mg. After 9 days of treatment at 300 mg, the patient developed severe renal hemorrhage, alveolar hemorrhage and gastrointestinal hemorrhage and subsequently died. A variety of bleeding events, tumor related and non-tumor related (e.g. epistaxis, gingival bleed, hematuria, hematochezia, melena) have also been observed across treated cohorts receiving up to 400 mg/d starting dose. However, evaluations of causality have been confounded by progressive bulky disease, prior surgery and concomitant medications such as nonsteroidal anti-inflammatory agents. In this study, at dose levels of 225 and 250 mg/d serious bleeding events have been limited to 1 of 14 patients. This patient experienced bleeding from the GE junction tumor site which resolved after study drug discontinuation.

The 250 mg daily dose of the compound of formula 2 selected as the recommended Phase 2 single agent dose was based on the safety and tolerability of this dose level in the Phase 1 trial in advanced disease, heavily pre-treated cancer subjects. The dose limiting toxicity of hypertension observed at the 300 mg dose level was not reported at the 250 mg dose level, serious bleeding events were limited to 1 gastro-esophageal tumor related event and diarrhea at the 250 mg dose level was tolerable with the addition of loperamide therapy as needed. In addition, drug concentrations at this dose were consistently above the lowest predicted human efficacious concentrations associated with anti-angiogenesis activity in preclinical models.

### EXAMPLE 5

A preliminary pharmacokinetic analysis has been performed on the compound of formula 2 concentration data from the patients in the Phase 1 study. In the higher dose cohorts in that study, blood specimens for pharmacokinetics were collected on Day 1 and 15 of Cycle 1 at the following times: just prior to dosing and at 0.5, 1, 2, 4, 6, 8, 12, 16, and 24 hours post-dose. Blood specimens for pharmacokinetics were also collected prior to and 2 hours after dosing on Days 3, 5, and 8 of Cycle 1. Among the 19 patients who received 250 mg QD dosing of the compound of formula 2, the mean Cmax (maximum observed plasma concentration) is 457 ng/mL, with a standard deviation of 240 ng/mL and the median Tmax (time of occurrence of Cmax) is 4 hours. Among these 19 patients, the 24-hour AUC values range from 2880 to 18700 ng·hr/mL. The values are distributed in a log-normal fashion with a geometric mean value of AUC of 7260 ng·hr/mL. The 1% value of the cumulative distribution of the data is 2000 ng·hr/mL and the 99% value is 25000 ng·hr/mL. Assuming dose proportionality, the 1% and 99% values of the cumulative distribution of 24-hour AUC values for doses other than 250 mg can be predicted by multiplying the respective values found for 250 mg by the ratio of the dose of interest divided by 250. For instance, the 99% value for a dose of 300 mg is predicted to be 25000 ng·hr/mL times 1.2 (300 divided by 250) which equals 30000 ng·hr/mL. The 1% value for a dose of 125 mg is predicted to be 2000 ng·hr/mL times 0.5 (125 divided by 250) which equals 1000 ng·hr/mL. Similar calculations provide predictions of the 1% (lower) and 99% (upper) values of the cumulative distribution of 24-hour AUC values for doses of 150 mg, 180 mg, 260 mg, and 280 mg.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A dosage form for administration to a mammal, the dosage form comprising a compound of formula 1: 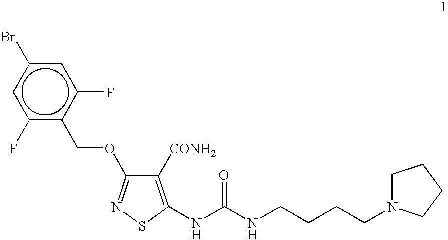
a pharmaceutically acceptable salt, solvate or prodrug thereof, or a mixture thereof, in an amount effective to provide a 24-hour area under the curve (AUC) value of no more than about 30000 ng·hr/mL of said compound, pharmaceutically acceptable salt, solvate or prodrug thereof, or a mixture thereof, or active metabolites thereof, after multiple daily (i.e., QD) administration to the mammal.

2. The dosage form of claim 1, wherein the pharmaceutically acceptable salt is a hydrochloride salt of the compound of formula 1.

3. The dosage form of claim 1, wherein the 24-hour AUC blood plasma value is from about 1000 ng·hr/mL to about 30000 ng·hr/mL.

4. The dosage form of claim 3, wherein the 24-hour AUC blood plasma value is from about 1200 ng·hr/mL to about 28000 ng·hr/mL.

5. The dosage form of claim 4, wherein the 24-hour AUC blood plasma value is from about 1440 ng·hr/mL to about 26000 ng·hr/mL.

6. The dosage form of claim 5, wherein the 24-hour AUC blood plasma value is from about 2000 ng·hr/mL to about 25000 ng·hr/mL.

7. The dosage form of claim 1, wherein the dosage form is an oral dosage form.

8. The dosage form of claim 1, wherein the dosage form is a tablet or a capsule.

9. A dosage form comprising a compound of formula 1: 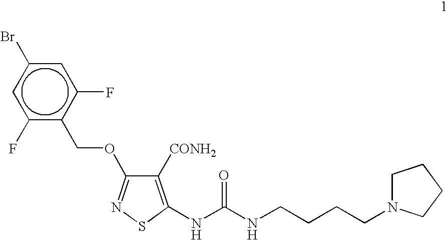
a pharmaceutically acceptable salt, solvate or prodrug thereof, or a mixture thereof, in an amount of no more than about 300 mg.

10. The dosage form of claim 9, wherein the pharmaceutically acceptable salt is a hydrochloride salt.

11-16. (canceled)

17. A method of treating a hyperproliferative disorder in a mammal which comprises administering to said mammal in need of such treatment a compound of formula 1 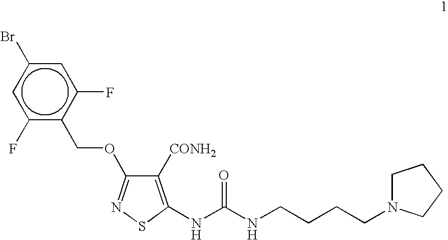
a pharmaceutically acceptable salt, solvate or prodrug thereof, or a mixture thereof, in an amount effective to provide a 24-hour area under the curve (AUC) value of no more than about 30000 ng·hr/mL of said compound, pharmaceutically acceptable salt, solvate or prodrug thereof, or a mixture thereof, or active metabolites thereof, after multiple daily (i.e., QD) administration to the mammal.

18-30. (canceled)

31. A method according to claim 17, further comprising administering to said mammal in need of such treatment, either simultaneously or sequentially with the compound of claim 1, a therapeutically effective amount of at least one compound selected from the group consisting of taxane derivatives and platinum coordination complexes selected from the group consisting of cisplatin, carboplatin, tetraplatin, taxotere and topotecan.

32-49. (canceled)
